(19) United States
(12) Patent Application Publication
(10) Pub. No.: US 2021/0128880 A1
(43) Pub. Date: May 6, 2021

(54) Catheter Securing Devices and Methods

(71) Applicant: Stephen Thomas, Hayden, AL (US)

(72) Inventor: Stephen Thomas, Hayden, AL (US)

(21) Appl. No.: 17/085,325

(22) Filed: Oct. 30, 2020

Related U.S. Application Data
(60) Provisional application No. 62/973,974, filed on Nov. 5, 2019.

(51) Int. Cl. A61M 25/02; A61M 25/00

(52) U.S. Cl. CPC A61M 25/02; A61M 2025/024; A61M 25/0017

(57) ABSTRACT

A device that has a head having an inside surface and a base integral with the head, the head smoothly transitioning to the base, the base comprising a first opening where the head transitions to the base and a lateral circular channel that travels from the first opening to a second opening on a bottom of the base. Further, the device has a clamp that clamps the base to a catheter.

### CROSS REFERENCE TO RELATED APPLICATION

This application claims priority to U.S. Provisional Patent Application No. 62/973,974 entitled Secure Cath and filed on Nov. 5, 2019, which is incorporated herein by reference.

### BACKGROUND

A catheter is a soft hollow tube, which is passed into the bladder to drain urine. Catheters are sometimes necessary for people, who for a variety of reasons, cannot empty their bladder in the usual way, i.e. passing urine into a toilet or urinal.

The main problems caused by urinary catheters are infections in the urethra, bladder or, less commonly, the kidneys. These types of infection are known as urinary tract infections (UTIs) and usually need to be treated with antibiotics. You can get a UTI from using either a short-term or a long-term catheter.

The infections of the urethra, which can translate into infections in the bladder or the kidneys, are often caused by bacteria. The bacteria enter the urethra through the genitalia that is exposed via the catheter to the surrounding environment.

### BRIEF DESCRIPTION OF THE DRAWINGS

The present disclosure can be better understood with reference to the following drawings. The elements of the drawings are not necessarily to scale relative to each other, emphasis instead being placed upon clearly illustrating the principles of the disclosure. Furthermore, like reference numerals designate corresponding parts throughout the several views.

FIG. 1 is a perspective view an exemplary catheter system comprising a catheter securing device in accordance with an embodiment of the present disclosure.

FIG. 2 is a perspective view an exemplary catheter securing device as shown in FIG. 1.

FIG. 3 is a front elevational view of the catheter securing device as shown in FIG. 2.

FIG. 4 is a side elevational view of the catheter securing device as shown in FIG. 2.

FIG. 5 is a top elevational view of the catheter securing device as shown in FIG. 2.

FIG. 6 is a bottom elevational view of the catheter securing device as shown in FIG. 2.

FIG. 7 is a cross-sectional side view of the catheter securing device as shown in FIG. 2.

### DETAILED DESCRIPTION

The present disclosure describes a catheter securing device that inhibits infection in a urethra, the bladder, and even the kidneys. The catheter securing device comprises a conical head that transitions to a cylindrical base. The catheter securing device comprises a clamp that secures the cylindrical base to the catheter. A catheter is inserted within a channel of the cylindrical base, travels through the conical head, and is inserted in a patient's urethra. The conical head of the catheter securing device creates a boundary between the environment and the patient's genitalia. This boundary ensures that bacteria is unexposed to the patient's genitalia and the patient's urethra. Thus, use of the catheter securing device reduces infections in the patient's urethra, bladder, and kidney.

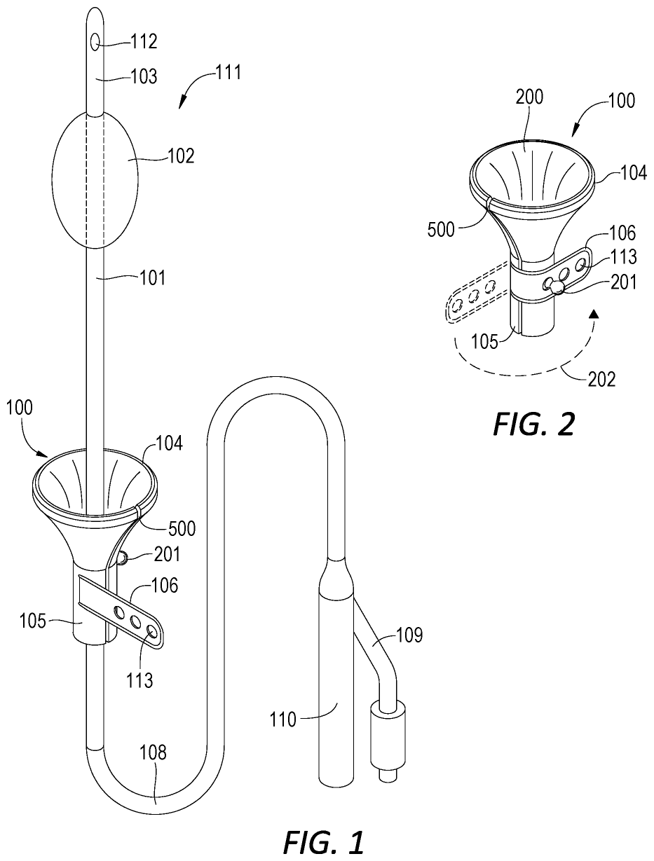

FIG. 1 is a perspective view of a catheter system 111 using a catheter securing device 100 in accordance with an embodiment of the present disclosure. The catheter system 111 comprises a catheter 101. The catheter 101 has a tip 103 that is inserted in a patient's bladder (not shown) to collect urine (not shown) through opening 112. The catheter 101 further comprises a balloon 102. When the catheter tip 103 is inserted in the patient's bladder, the balloon may be filled via a pump with water to ensure that the tip 103 remains inserted in the patient's bladder.

The catheter 101 is integral with and coupled to tubing 108. Urine from the patient's bladder travels through the tubing 108 out a receptacle 110 and to a bag (not shown) for collection. A receptacle 109 is coupled to the tubing 108 to pump water from a bag (not shown) to the balloon 102. Note that water may be bi-directionally pumped to and from the balloon 102.

The catheter securing device 100 is coupled to the catheter 101, which is described further herein. The catheter securing device 100 comprises a conical head 104 that smoothly transitions to a cylindrical base 105. A lateral gap 500 begins at a top of the conical head and continues to the bottom of the cylindrical base 105. Further, the catheter securing device 100 further comprises a flap 106. The flap 106 comprises a plurality of openings 113. Thus, to secure the catheter securing device 100 to the catheter 101, the flap 106 is wrapped around the cylindrical base 105 until one of the openings 113 fits over a protruding stop 201. This tightens the catheter securing device 100 around the catheter 101 by closing the gap 500.

Further, the conical head 104 fits against the patient's genitalia. In one embodiment, antimicrobial material, e.g., antimicrobial foam, may be applied to the inside of the conical head and the inside of the cylindrical base to prevent growth of bacterial.

In effect, the conical head 104 covers the patient's genitalia. Thus, the bacteria are less likely to be exposed to the patient's genitalia. Therefore, the patient is less likely to contact a bacterial infection when using the catheter 101.

FIG. 2 is a perspective view of the catheter securing device 100. As noted, the catheter securing device 100 comprises a conical head 104 that smoothly transitions to the cylindrical base 105. Further, the flap 106 wraps around the cylindrical base 105 and is coupled to the protruding stop 201.

Note that there are three openings 113 in the flap 106 so that the catheter securing device 100 may be used with catheters with differing diameters. Further, the arrow 202 indicates that the flap 106 is wrapped around cylindrical base 105 and is coupled to the protruding stop 201 via one of the openings 113. This secures the catheter securing device 100 to the catheter 101 (FIG. 1). As the flap 106 is wrapped tighter around the cylindrical base 105, the gap 500 closes, thereby securing the catheter securing device 100 to the catheter 101.

When secured to the catheter 101 (FIG. 1), the conical head 104 covers the patient's genitalia. Thus, an inside surface 200 may be in contact with the patient's genitalia.

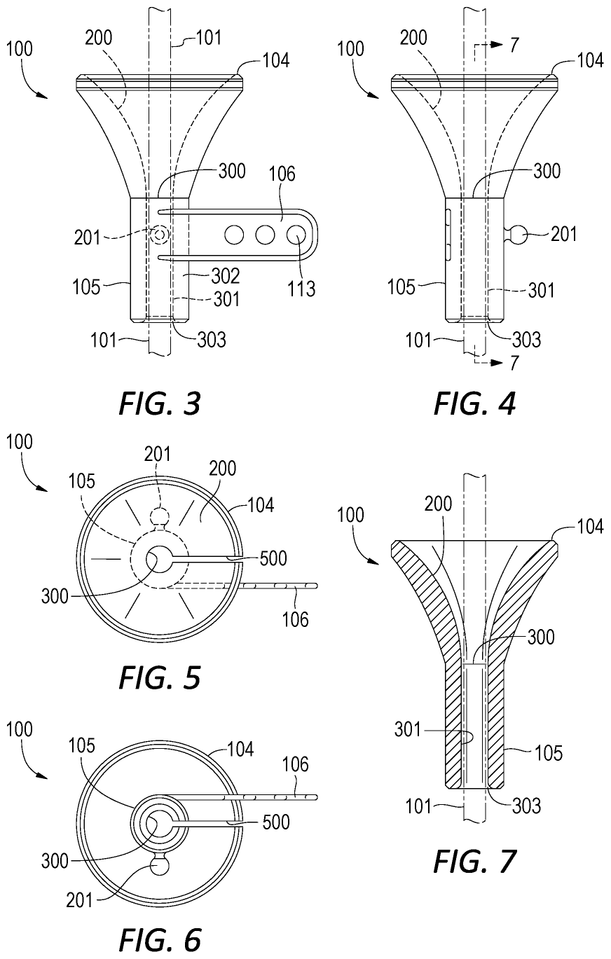

FIG. 3 is a front elevational view of the catheter securing device 100. The catheter securing device 101 comprises the conical head 104 that smoothly transitions to the cylindrical base 105. The flap 106 is coupled to and extends from an outside surface 302 of the cylindrical base 105.

The catheter 101 (FIG. 1) is inserted in the patient's urethra and ultimately the bladder. The catheter 101 travels through an opening 300 in the cylindrical base 105. Further, the catheter 101 travels through a cylindrical channel 301 in the cylindrical base 105, and the catheter 101 exits from a bottom opening 303 in the cylindrical base 105.

Once the catheter 101 is inserted in the catheter securing device 100, a user, e.g., a nurse or doctor, grasps the flap 106. The user pulls the flap 106 around the cylindrical base 105 and couples the flap 106 to the protruding stop 201. When the flap 106 is wrapped around the cylindrical base 105 and coupled to the protruding stop, the lateral gap 500 (FIG. 1) closes thereby tightening the catheter securing device 100 to the catheter 101.

As described hereinabove, when the catheter securing device 100 is coupled to the catheter 101, the conical surface 200 rests against or covers the patient's genitalia. When the genitalia are covered by the conical head 104, the patient is less likely to contract a bacterial infection in his/her urethra, bladder, and kidneys.

FIG. 4 is a side elevational view of the catheter securing device 100. The catheter securing device 101 comprises the conical head 104 that smoothly transitions to the cylindrical base 105. The flap 106 (FIG. 1) is coupled to and extends from an outside surface 302 of the cylindrical base 105.

The catheter 101 (FIG. 1) is inserted in the patient's urethra and ultimately the bladder. The catheter 101 travels through an opening 300 in the cylindrical base 105. Further, the catheter 101 travels through a cylindrical channel 301 in the cylindrical base 105, and the catheter 101 exits from a bottom opening 303 in the cylindrical base 105.

Once the catheter 101 is inserted in the catheter securing device 100, a user, e.g., a nurse or doctor, grasps the flap 106. The user pulls the flap 106 around the cylindrical base 105 and couples the flap 106 to the protruding stop 201. When the flap 106 is wrapped around the cylindrical base 105 and coupled to the protruding stop, the lateral gap 500 (FIG. 1) closes thereby tightening the catheter securing device 100 to the catheter 101.

As described hereinabove, when the catheter securing device 100 is coupled to the catheter 101, the conical surface 200 rests against or covers the patient's genitalia. When the genitalia are covered by the conical head 104, the patient is less likely to contract a bacterial infection in his/her urethra, bladder, and kidneys.

FIG. 5 is a top view of the catheter securing device 100. The catheter securing device 100 comprises the conical head 104 having a conical inside surface 200. The conical inside surface 200 contacts the patient's genitalia.

Further, the conical head 104 smoothly transitions to the cylindrical base 105. The cylindrical base 105 comprises the opening 300. In the opening 300 is the lateral gap 500. In operation, the flap 106 is wrapped around the cylindrical base 105 and coupled to the protruding stop 201 thereby closing the lateral gap 500 and tightening the catheter securing device 100 to the catheter 101 (FIG. 4).

The inside surface 200 rests against the patient's genitalia when the catheter securing device 100 is installed. The conical head 104 protects the patient's genitalia from bacteria. Thus, the catheter securing device 100 protects the patient from bacterial infections in his/her urethra, bladder, and kidneys.

FIG. 6 is a bottom view of the catheter securing device 100. The catheter securing device 100 comprises the conical head 104 having a conical inside surface 200 (FIG. 5). The conical inside surface 200 contacts and protects the patient's genitalia.

Further, the conical head 104 smoothly transitions to the cylindrical base 105. The cylindrical base 105 comprises the opening 300. In the opening 300 is the lateral gap 500. In operation, the flap 106 is wrapped around the cylindrical base 105 and coupled to the protruding stop 201 thereby closing the lateral gap 500 and tightening the catheter securing device 100 to the catheter 101 (FIG. 4).

The inside surface 200 rests against the patient's genitalia when the catheter securing device 100 is installed. The conical head 104 protects the patient's genitalia from bacteria. Thus, the catheter securing device 100 protects the patient (not shown) from bacterial infections in his/her urethra, bladder, and kidneys.

FIG. 7 is a cross-sectional view of the catheter securing device 100. The catheter securing device 100 comprises the conical head 104 having a conical inside surface 200 (FIG. 5). The conical inside surface 200 contacts and protects the patient's genitalia.

The conical head 104 smoothly transitions to the cylindrical base 105. In this regard, the inside surface 200 of the conical head 104 slowly decreases in diameter until it transitions to opening 300.

The catheter 101 is inserted into the patient's urethra (not shown). The catheter 101 travels through the conical head 104 and into the opening 300. The catheter 101 travels through the cylindrical base 105 and exits the cylindrical base 105 through the opening 303.

The flap 106 (FIG. 6) is wrapped around the cylindrical base 105 and is coupled to the projection 201 (FIG. 6). When the flap 106 is coupled to the projection 201, the lateral gap 500 (FIG. 6) closes thereby tightening the catheter securing device 100 to the catheter 101.

When installed, the inside surface 200 of the conical head 104 contacts and protects the patient's genitalia from bacteria. Thus, the catheter securing device 100 protects the patient (not shown) from bacterial infections in his/her urethra, bladder, and kidneys.

1. A device, comprising:
a head having an inside surface;
a based integral with the head, the head smoothly transitioning to the base, the base comprising a first opening where the head transitions to the base and a lateral circular channel that travels from the first opening to a second opening on a bottom of the base;
a clamp that clamps the base to a catheter.

2. The device of claim 1, wherein the head is a conical head, and the inside surface is a conical inside surface.

3. The device of claim 1, wherein the base is a cylindrical base and the catheter travels through the channel to the second opening.

4. The device of claim 1, wherein the head is a conical head, and the base is a cylindrical base and the conical head smoothly transitions to the cylindrical base.

5. The device of claim 1, wherein the clamp comprises a flap coupled to an outside surface of the base.

6. The device of claim 1, wherein a projection is coupled to the outside surface of the base.

7. The device of claim 1, wherein the head and the base comprise a lateral gap that extends from a top of the head to a bottom of the base.

8. The device of claim 1, wherein when the flap is coupled to the projection, the gap closes thereby securing the base to the catheter.

9. The device of claim 1, wherein the first opening is configured for receiving a catheter and the catheter travels through a channel of the base and exits the second opening.

10. The device of claim 1, wherein when the head and the base are coupled to a catheter, the head protects a person's genitalia from bacteria.
